(19) United States
(12) Patent Application Publication
(10) Pub. No.: US 2019/0224292 A1
(43) Pub. Date: Jul. 25, 2019

(54) Methods of Augmenting Tumor Vaccine Efficacy through Endothelial Targeting

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/250,897

(22) Filed: Jan. 17, 2019

Related U.S. Application Data
(60) Provisional application No. 62/620,045, filed on Jan. 22, 2018.

(51) Int. Cl. A61K 39/00; A61P 35/00; A61K 39/39; A61K 9/00

(52) U.S. Cl. CPC A61K 39/001102; A61P 35/00; A61K 39/39; A61K 45/06; A61K 2039/5152; A61K 2039/5154; A61K 2039/5158; A61K 9/0019

(57) ABSTRACT

Disclosed are means, methods, and compositions of matter useful for augmentation of a vaccine induced antitumor immune response through immunological targeting of tumor endothelium. In one embodiment a cancer antigen specific vaccine is combined with an endothelial targeting vaccine in a manner to facilitate release of tumor antigens as a result of endothelial destruction. The released tumor antigens serve to amplify immune response induced by the cancer antigen specific vaccine. In one embodiment an endogenous cancer vaccine is utilized as a source of immunization. The endogenous cancer vaccine may be cancer cells induced to die locally such as by means of: hyperthermia, irradiation, oncolytic virus exposure, and embolization.

### CROSS-REFERENCE TO RELATED APPLICATION

This application claims the benefit of and priority to U.S. Provisional Patent Application No. 62/620,045, filed on Jan. 22, 2018, the contents of which are incorporated herein by reference in its entirety.

### BACKGROUND

In the last Century, the focus of cancer research in general has been the development of therapies that not only destroy, inhibit, or block progression of primary tumors, but also suppress micrometastatic and metastatic progeny of the primary tumor from seeding the patient. Despite extensive research into the disease, effective means of treating the majoring of cancers at present are elusive to the medical community. Although limited success is achieved using the current standard therapies: chemotherapy, radiation therapy, and surgery; each therapy has inherent limitations. Chemotherapy and radiation therapy cause extensive damage to normal, healthy tissue such as bone marrow, intestinal cells, and even neuronal cells, despite efforts to target such therapy to abnormal tissue (e.g., tumors). Surgery is in many cases effective in removing masses of cancerous cells; however, it cannot always ensure complete removal of affected tissue nor are all tumors in an anatomical location amenable to surgical removal. Furthermore, subsequent to surgical removal, the problem of metastasis and reoccurrence remains unresolved.

The use of the immune system for killing cancer is an area of active investigation. Immunological control of neoplasia is suggested by: A) Evidence of longer survival of patients with a variety of cancers who possess a high population of tumor infiltrating lymphocytes; B) The fact that immune suppressed patients develop cancer at a much higher frequency in comparison to non-immune suppressed individuals; and C) In some very particular situations immunotherapy of cancer is clinically effective [6]. While cancer immunotherapy offers the possibility of inducing remission and control of both the primary tumor mass, as well as micrometastasis, several drawbacks exist. The most significant one is that in many situations immunotherapy is either not feasible or associated with a variety of toxicities. Various types of immunotherapies for cancer have been attempted, including: a) systemic cytokine administration; b) gene therapy; c) allogeneic vaccines; d) autologous vaccine; e) heat shock protein vaccines; f) dendritic cell vaccines; g) tumor infiltrating lymphocytes; h) administration of T cells in a lymphodepleted environment; and i) nutritional interventions. Although each of the approaches contains significant advantages and drawbacks, none of them simultaneously meet the criteria of reproducible efficacy, availability to the mass population, or tumor selectivity/specificity.

The limitations of many immunotherapeutic approaches to cancer is that tumor antigens are either not clearly defined, or in situations where they are defined, the tumor either mutates to lose expression of such antigens, or the antigen-specific vaccine is only applicable to patients with a certain major histocompatibility complex haplotype. The circumvention of this problem has been attempted using autologous vaccines, however in many cases this is an expensive and difficult procedure.

### DESCRIPTION OF THE INVENTION

The invention teaches the utilization of endothelial targeting vaccines as a means of augmenting efficacy of cancer antigen specific vaccines. In one embodiment the invention teaches that administration of ValloVax, a placental endothelium-based vaccine described in the following references, is capable of sensitizing tumors to treatment with cancer vaccines, the cancer vaccines comprising of either peptide vaccines, protein vaccines, cellular vaccines, or endogenous vaccines. Without being bound to theory, the cancer endothelial targeting vaccines are capable of specifically inducing inactivation of tumor endothelial mediated lymphocyte death, thus allowing for cancer killing T cells to specifically enter the tumor and mediate tumor cell death. As substitution for ValloVax, other types of endothelial progenitor cells (EPC) may be used to stimulate immunity to tumor endothelium. The EPC, in one embodiment are a population of cells comprising cells having the surface marker CD44, cells having the surface marker CD13, cells having the surface marker CD90, cells having the surface marker CD105, cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s. The EPC may be treated in a manner to mimic the tumor microenvironment, specifically, they may be grown under the acidic conditions in the tumor microenvironment and are incorporated by reference. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation, and glutamine deprivation.

“Marker” and “Biomarker” are used interchangeably to refer to a gene expression product that is differentially present in samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

“Immunoassay” is an assay that uses an antibody to specifically bind an antigen (e.g., a marker). The immunoassay is characterized using specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting the expression, in more general terms, intervening in the expression, for instance by affecting the expression of a gene encoding that protein.

In one embodiment, EPCs refer to endothelial colony-forming cells (ECFCs) and their progenitor cell capacities were characterized as described (Wu, Y et al., J Thromb Haemost, 2010; 8:185-193; Wang, H et al., Circulation research, 2004; 94:843 and Stellos, K et al., Eur Heart J., 2009; 30:584-593). Briefly, human blood was collected from healthy volunteer donors. All volunteers had no risk factors of CVD including hypertension, diabetes, smoking, positive family history of premature CVD and hypercholesterolemia, and were all free of wounds, ulcers, retinopathy, recent surgery, inflammatory, malignant diseases, and medications that may influence EPC kinetics. After dilution with HBSS (1:1), blood was overlaid onto Histopaque 1077 (Sigma-Aldrich Co. LLC, St. Louis, Mo.) in the ratio of 1:1 and centrifuged at 740 g for 30 minutes at room temperature. Buffy coat MNCs were collected and centrifuged at 700 g for 10 minutes at room temperature. MNCs were cultured in collagen type I (BD Bioscience, San Diego) (50 m/ml)-coated dishes with EBM2 basal medium (Lonza Inc., Allendale, N.J.) plus standard EGM-2 SingleQuotes (Lonza Inc., Allendale, N.J.) that includes 2% fetal bovine serum (FBS), EGF (20 ng/ml), hydrocortisone (1 μg/ml), bovine brain extract (12 μg/ml), gentamycin (50 m/ml), amphotericin B (50 ng/ml), and epidermal growth factor (10 ng/ml).

Colonies appeared between 5 and 22 days of culture were identified as a well-circumscribed monolayer of cobblestone-appearing cells. ECFCs with endothelial lineage markers expression, robust proliferative potential, colony-forming, and vessel-forming activity in vitro are defined as EPCs as described (Wang, H et al., Circulation research, 2004; 94:843 and Stellos, K et al., Eur Heart J., 2009; 30:584-593). Passage 4 to 6 EPCs were used for experiments. For a brief characterization, endothelial phagocytosis function was confirmed by incubating EPC in 4-well chamber slide with 1, 1-dioctadecyl-3, 3, 3, 3-tetramethylindocarbocyanine (DiI)-labeled acetylated low-density lipoprotein (acLDL) (Biomedical Technologies, Inc., Stoughton, Mass.) (5 m/ml) at 37° C. for 1 h, washed 3 times for 15 min in PBS, and then fixed with 2% paraformaldehyde for 10 min. Cells were then incubated with FITC conjugated UEA-1 (Ulex europaeus agglutinin) (10 m/ml) (Sigma-Aldrich Corporation, St. Louis, Mo.) for 1 h at room temperature, which is capable of binding with glycoproteins on the cell membrane to allow visualization of the entire cell. Cell integrity was examined by nuclear staining with DAPI (100 ng/ml). After staining, cells are imaged with high-power fields under an inverted fluorescent microscope (Axiovert 200, Carl Zeiss, Thornwood, N.Y.) at 200.times. magnification and quantified using Image J software.

The EPC may be identified by means by selecting for cells expressing genes, wherein genes are at least one gene selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYBSB, DLL4, DUSPS, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1). The EPC may be purified from a variety of sources, including peripheral blood, placental cells, cord blood, umbilical cord, adipose tissue and bone marrow.

In another embodiment, EPC are characterized by expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYBSB, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYBSB, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

Conversely, the step of increasing the number of activated endothelial progenitor cells comprises increasing in the endothelial progenitor cells in the blood of the subject the expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYBSB, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

The generation of EPC and EPC-derived endothelial cells is disclosed in the invention through culture of EPC or EPC-derived endothelial cells in conditions which resemble the tumor microenvironment. One such condition is exposure to ionic concentrations which resemble the tumor microenvironment. It is known that tumors contain areas of cellular necrosis, which are associated with poor survival in a variety of cancers. A study showed that necrosis releases intracellular potassium ions into the extracellular fluid of mouse and human tumors, causing profound suppression of T cell effector function. Elevation of the extracellular potassium concentration ([K⁺]ₑ) impairs T cell receptor (TCR)-driven Akt-mTOR phosphorylation and effector programs. Potassium-mediated suppression of Akt-mTOR signaling and T cell function is dependent upon the activity of the serine/threonine phosphatase PP2A. Although the suppressive effect mediated by elevated [K⁺]_{c} is independent of changes in plasma membrane potential (Vₘ), it requires an increase in intracellular potassium ([K⁺]ᵢ). Accordingly, augmenting potassium efflux in tumor-specific T cells by overexpressing the potassium channel Kᵥ1.3 lowers [K⁺]ᵢ and improves effector functions in vitro and in vivo and enhances tumor clearance and survival in melanoma-bearing mice. In one embodiment the invention teaches the use of culture conditions similar to those associated with necrotic tissue as a means of modifying EPC and EPC-derived endothelial cells to render the cells similar to tumor endothelial cells. In other embodiments of the invention EPC or EPC-derived endothelial cells are cultured under conditions of free adenosine similar to those found in tumor cells. Numerous publications report concentrations found in tumors and several are incorporated by reference. In one embodiment of the invention EPC or endothelial cells derived thereof are cultured with enzymes known to induce production of adenosine locally in a manner similar to that found in the tumor microenvironment. Enzymes, or ectoenzymes useful for the practice of the invention include CD39, and CD73, which are described in the associated references and incorporated herein.

It is known that the tumor endothelium acts as a protective barrier to the immune system from attacking the cancer. In the practice of the invention, the tumor endothelial targeting vaccines are used to reduce or substantially abrogate the about of the tumor endothelium to protect the tumor from infiltrating immune cells.

One example of how the cancer endothelium protects the tumor is through expression of FasL. FasL was discovered and cloned by Suda et al in 1993 as a member of the tumor necrosis factor family, which was subsequently showed to induce apoptosis in various cells expressing Fas, such as T lymphocytes. It is known that FasL and Fas, play a key role in the regulation of apoptosis in the immune system. FasL acts as a cytotoxic effector molecule to Fas-expressing malignant tumor cells; however, it has recently been suggested that FasL also acts as a possible mediator of tumor immune privilege. In a recent study, FasL expression in glioblastoma associated endothelial cells were examined by Western blotting and immunohistochemistry. In addition, quantitative analysis of T-cell infiltration in these tumors was performed. FasL expression was seen in all cell lines and in 9 of 14 specimens by Western blotting and immunohistochemistry. The distribution of FasL was recognized in the tumor vascular. Both types of FasL expression were associated with a significant reduction (p<0.05) in T-cell infiltration when compared with FasL-negative areas within the same tumor or FasL-negative specimens.

Since T-cell apoptosis could be induced by FasL-expressing tumor endothelial cells, authors that apoptosis induction by FasL expressed on tumor cells and/or vascular endothelium might be one mechanism for T-cell depletion in astrocytic tumor tissues. Thus, it appears that prevention of T cells from entering tumors is mediated in part by the barrier posed by the blood vessel containing death ligands. The importance of FasL maintaining immune privilege has been observed in physiological situations. For example, immune privilege of the eye [83-88], the nucleus pulposus of the intravertebral disc, the testis, the blood brain barrier, and the placenta, is associated with expression of FasL. In another study, investigators sought to determine T cell presence in TIL, and the ratio of CD8+ and CD4+ T cell subsets in particular, can correlate with tumor prognosis in some tumors, although the significance of such infiltration into glioma is controversial. However, gliomas represent a lower extreme in their extent of T cell infiltration and are thus useful in assessing factors that can decrease T cell presence within tumor tissue. Fas ligand, a pro-apoptotic cell surface protein, may play a key role in reduction of T cells in tumor tissue. To assess the level of FasL expression on brain tumor endothelium and to correlate this with relative levels of CD4+ and CD8+ T cell subsets in TIL from brain tumors. CD3+, CD4+, and CD8+ cells were quantified in fresh TIL by flow cytometry. Paraffin embedded sections of tumors, including meningiomas and gliomas as well as extracranial malignancies, underwent immunohistochemical staining for FasL and Von-Willebrand's factor (Factor VIII) to determine expression levels of endothelial FasL. FasL expression was high in aggressive intracranial malignancies compared to more indolent neoplasms and correlated inversely with CD8+/CD4+ TIL ratios in all tumor classes combined (ANOVA, p<0.05). Low levels of T cells within TIL, as well as low CD8+/CD4+ TIL ratios appear to be a property of parenchymal tumor presence. Together with the inverse correlation seen between FasL expression and CD8+/CD4+ TIL ratios, the high levels of endothelial FasL expression in gliomas suggests that FasL decreases T cell presence in brain tumors in a subset-selective manner, thus contributing to glioma immune privilege.

It is known in the literature that patients in which infiltration of T cells and NK cells (tumor infiltrating lymphocytes (TILs)) is observed, that these patients possess a better prognosis as compared to patients without tumor infiltrating lymphocytes. Thus, in one embodiment, endothelial targeting vaccines are utilized as a means of augmenting ability of lymphocytes to enter the tumor. TILs have been noticed in a variety of tumors and are correlated with a favorable prognosis in certain cancers including liver carcinoma, melanoma, bladder cancer, colorectal cancer, and ovarian cancer. It is the belief of many tumor immunologists that TILs infiltrate tumors to induce their eradication, however, this does not occur in vivo because tumor-secreted immunosuppressive factors inhibit immune activation. TIL therapy involves surgically extricating a tumor mass, separating the TILs from the tumor cells on a density gradient, expanding the lymphocytes in immunostimulatory in vitro conditions and reinfusing the activated killer cells back into the patient. Mouse models contrasting the antitumor efficacy of TIL therapy to LAK therapy showed that TIL therapy had approximately a one hundred-fold greater tumoricidal effect. A possible reason why TILs had an augmented tumor eradicating effect is that this therapy activates only lymphocytes that have recognized the tumor and are reacting to it. In the clinic, results using TIL have been fair, with reproducible responses in approximately 20% of melanoma patients. A means of augmenting the efficacy of TILs is to enhance their killing potential by transfecting them with cDNA to TNF. Thus, in one embodiment of the invention, tumor endothelial targeting vaccines are utilized to overcome cancer endothelial mediated immune evasion of the tumor, which potentiates the ability of the vaccine induced T cells to kill tumors.

Numerous mean of stimulating immunity to tumor associated endothelial cells are known in the art. In one embodiment, growth factors, growth factor receptors, or antigens associated with tumor endothelial cells are chosen for production of a vaccine. Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin, endosialin, and xenogeneic FGF2R, VEGF, VEGF-R2, MMP-2, and endoglin. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities.

For example, in one study description of optimization of endothelial cell-based vaccines was described. The authors of the study utilized human umbilical vein endothelial cells (HUVEC), which were prepared in different ways. Specifically tested was 1) paraformaldehyde-fixed HUVEC; 2) glutaraldehyde-fixed HUVEC; 3) HUVEC lysate and; 4) live HUVEC, these four commonly used antigen forms were used to prepare vaccines named Para-Fixed-EC, Glu-Fixed-EC, Lysate-EC, and Live-EC respectively. The investigators showed that Live-EC exhibited the most favorable anti-tumor growth and metastasis effects among the four vaccines in both H22 hepatocellular carcinoma and Lewis lung cancer models. High titer anti-HUVEC antibodies were detected in Live-EC immunized mice sera, and the immune sera of Live-EC group could significantly inhibit HUVEC proliferation and tube formation. Moreover, T cells isolated from Live-EC immunized mice exhibited strong cytotoxicity against HUVEC cells, with an increasing IFN-γ and decreasing Treg production in Live-EC immunized mice. Finally, CD31 immunohistochemical analysis of the excised tumors verified a significant reduction in vessel density after Live-EC vaccination, which was in accordance with the anti-tumor efficiency. Taken together, all the results proved that live HUVEC was the most effective antigen form to induce robust HUVEC specific antibody and CTL responses, which are known to lead to the significant inhibition of tumor growth and metastasis [136]. Accordingly, in one embodiment of the invention, live HUVEC cells are utilized as a vaccine for stimulation of immunity towards tumor endothelial cells, wherein the invention teaches that this stimulation of immunity results in sensitization of the tumor to conventional cancer vaccines that induce activation of T cells or B cells. Furthermore, the invention teaches means of overcoming the immune privileged state of the tumor endothelium by means of selectively inhibiting the tumor endothelial immune suppressive state. Elimination of immune suppressive state can be accomplished by induction of killing of tumor endothelium but can also be accomplished by blocking of suppressive factors, proteins, or peptides found on the tumor endothelium. For example, in one embodiment, the vaccination with tumor endothelium targeting immunogens can lead to antibodies to molecules such as FasL, which block the ability of the FasL on the tumor endothelium to induce killing of T cells attempting to infiltrate the tumor. Means of inactivation of immune suppressive molecules found on tumor endothelium include antibody blockade of function, generation of coagulation on the surface of the tumor endothelium, as well as complement activation on the surface of the tumor endothelium.

Addition of various adjuvants may be used to increase immunity of vaccines whose role is to stimulate immunity to tumor endothelium. Various adjuvants are known in the art, including various agonists of toll like receptors. Particular adjuvants include lipopolysaccharide an activator of TLR-4, Poly IC, a TLR-3 agonist, imiquimod a TLR-7 agonist, and CpG motifs such as TLR-9. Other adjuvants useful for the practice of the invention include Freund's Complete Adjuvant, Freund's Incomplete Adjuvant, BCG, and loading on antigen presenting cells. In one embodiment adjuvants are selected from a group comprising of: Cationic liposome-DNA complex JVRS-100, aluminum hydroxide, aluminum phosphate vaccine, aluminum potassium sulfate adjuvant, Alhydrogel, ISCOM(s), Freund's Complete Adjuvant, Freund's Incomplete Adjuvant, CpG DNA Vaccine Adjuvant, Cholera toxin, Cholera toxin B subunit liposomes, Saponin, DDA, Squalene-based Adjuvants, Etx B subunit, IL-12, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, thalidomide, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El12K of Cholera Toxin mCT-E112K, and Matrix-S.

One type of antigen presenting cell useful as an adjuvant for the practice of the invention are dendritic cells. Numerous means of generating DC are described in the art. In one embodiment peripheral blood mononuclear cells (PBMC) are extracted and subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7.

In some embodiments, augmentation of endogenous cellular vaccines is performed by stimulating immunity to tumor endothelium. The immunity towards the tumor endothelium is aimed to allow a sensitization of the tumor to T cells. In other embodiments, targeting of the tumor endothelium is performed to overcome the ability of the endothelium to protect the tumor.

In one embodiment of the invention, the use of endogenous release of tumor antigens is used as a source of tumor antigens. One method of inducing localized tumor cell death is transarterial chemoembolization (TACE), or otherwise defined as transcatheter chemoembolization, is a clinical procedure used primarily for treating primary and secondary liver cancer. TACE is usually employed when standard therapy has failed or is known to be ineffective. TACE combines the advantages of intra-arterial chemotherapy, with the fact that embolization of the portal artery induces a preferential “starvation” of the tumor while sparing non-malignant hepatic tissue. Specifically, it is established that intra-arterial delivery of chemotherapy to the liver results in a tenfold higher intratumoral concentration as compared to administration through the portal vein. This is due in part to the observation that both primary and secondary liver tumors derive their blood supply preferentially from the hepatic artery. Anecdotal evidence suggested that embolization caused by thrombosis of the catheter during delivery of intraarterial chemotherapy as beneficial for inducing an improved tumor response. This prompted investigators to use surgical ablation [140] or angiographic embolization to induce localized necrosis. Unfortunately, this approach, in absence of chemotherapy caused little effect on long-term survival. Therefore the advantages of TACE is that both localized delivery of chemotherapy to the tumor occurs, while at the same time, the tumor blood flow is embolized, causing local tumor necrosis. These advantages are used for the practice of the invention, in which combination of TACE with cancer endothelial cell vaccination is performed.

The stimulation of cancer immunity is a result, in one embodiment, by release of tumor cell antigens rom dying cancer cells. Globally speaking, apoptotic cell death is associated with anti-inflammatory and in some cases tolerogenesis, whereas necrotic cell death is perceived by the immune system as a “danger signal” and is associated with immune activation. Specific examples of the anti-inflammatory aspects of apoptotic cell death include: the production of IL-10 by apoptotic monocytes; suppression of inflammatory cytokines by apoptotic bodies in vitro, observations that administration of apoptotic but not necrotic cell bodies can actually endow macrophages with active immune suppressive properties; and clinically administered apoptotic blood cells have been demonstrated successful for treatment of inflammation associated with advanced heart failure in a recent Phase II trial. Conversely, cellular necrosis is associated with release of a variety of innate immune activation signals such as heat shock proteins, HMGB1, mRNA with endogenous secondary structures, and even DNA complexed with endogenous factors such as natural antibodies. Therefore, the induction of cellular necrosis caused by TACE induces a release of tumor antigens, which is picked up by the immune system. The release of tumor antigens in such situations is reported in the literature, however taking advantage of this antigen release in the therapeutic context has not been accomplished to date. Although the in the case of hepatocellular carcinoma, tumor itself, and host cells infiltrating the tumor are known to be immune suppressive, the microenvironment in which TACE induces cellular necrosis is also normally immune suppressive.

It is known that intrahepatic administration of antigens results in systemic immune deviation towards weak cellular immunity. For example, it was demonstrated that administration of donor cells into the hepatic circulation resulted in prolonged, donor specific, graft acceptance in various models of transplantation. The localized immune suppressive effects of the liver are known to the transplant clinician in that liver transplant recipients require a lower degree of immune suppression as compared to other organs. Additionally, in various rodent strain combinations hepatic grafts are spontaneously accepted, while cardiac or renal are rejected. At a cellular level this is explained by the presence of immature hepatic DC, the tolerogenic potential of liver sinusoidal endothelial cells, as well as natural killer T cells with a predisposition for releasing IL-4. Based on this, a release of tumor antigens within the hepatic microenvironment is postulated to cause a Th2, or immune regulatory shift, thereby not only failing to initiate protective immunity towards micrometastasis, but in some cases maybe even increasing the rate of tumor growth, through the phenomena of “tumor enhancement” described by Prehn. Accordingly, it is the object of the current invention to stimulate Th1 immunity, which is cell based, and avoid antibody-based immunity to the tumor cells.

One specific embodiment of the invention involves modification of the TACE procedure in order to induce a systemic anti-tumor immunological effect. Specifically, patients are selected to meet the criteria for TACE. The criteria include: a) Adequate hepatic function; b) Patent portal vein circulation (confirmed during the venous phase of celiac or superior mesenteric angiogram); and c) Adequate renal function. Generally, only patients without cirrhosis or in Child group A or B disease are considered, however depending on experience of the practicing physician other groups may be included in the procedure as discussed by Shah et al. The TACE procedure may be performed either using a selective or superselective means. Patients selected to undergo the procedure receive 10 mg of phytonadione intravenously prior to the procedure (the intravenous injection should be administered slowly). Femoral catheterization and positioning of the catheter is performed. Premedication is with Lorazepam (Wyeth Laboratories, UK) 0.25 mg/kg orally 1 hour before the procedure to counter anxiety. An intra-arterial injection of 30-40 mg of 1% lidocaine is used for analgesia.

The following ingredients are made into an emulsion by repeatedly emptying and filling a syringe over 10 minutes: 10 mL of Lipiodol Ultrafluid (Mallinckrodt Medical, UK), 5 mL Omnipaque 300 (Amersham Health, UK; water-soluble contrast aids in emulsifying the mixture), 50 mg doxorubicin and clinical grade Poly (IC) stabilized with carboxymethylcellulose at a concentration between 0.025 mg/m² to 12 mg/m², preferably at a concentration of 0.2 mg/m². Intraarterial injection is administered under direct visualization to prevent reflux into gastroduodenal or splenic vessels. Embolization is performed with Ultra Ivalon 250-400 μm (Laboratories Nycomed SA). Intravenous cefuroxime (750 mg) and metronidazole (500 mg) are administered 3 times per day for 5 days. These antibiotics are given as prophylaxis against septicemia and liver abscess formation. Subsequent to administration patients are admitted to a high-dependency ward and should be mobilized after 6 hours of bedrest. Postoperative analgesia is administered if and when required by the patient. Patients also receive ranitidine (an H2 antagonist) intravenously 3 times per day until they begin eating. Patients are discharged home after 5 days or when their systemic symptoms begin resolving. In order to monitor success of the procedure nonenhanced and enhanced CT examinations are performed 10-14 days following embolization. Furthermore, alpha-fetoprotein levels are evaluated at the 6-week outpatient review. If the TACE procedure is successful (>50% lipiodol uptake in necrotic tumor demonstrated on the postprocedural CT scan), the embolization is repeated in 6-8 weeks. Immunological monitoring is performed by assessing levels of interferon alpha production using ELISA during the 12, 24, and 72-hour time periods. Additionally, DTH, cellular and antibody responses are measured using pre-defined antigens representative of the tumor type.

A variety of chemotherapeutic agents can be used in practicing the invention. Specifically, chemotherapeutic agents which induce upregulation of costimulatory molecules are preferred. One example of such an agent is melphalan, which induces expression of CD80 on both tumor cells, as well as non-tumor B cells. In addition, a wide variety of chemotherapeutic agents are known in the art. These include: alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′, 2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE® Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; and capecitabine. TACE-modification procedure was presented as an example of illustrating the disclosed invention.

Additionally, modifications may be made to increase efficacy of anti-tumor response being mediated. Particularly, a wide variety of agents can be administered to the patient prior to the TACE procedure in order to increase general immunological status, and specifically, T cell, NK cell, and NKT cell functions. One particular modification may involve the administration of an anti-oxidant capable of reversing immune suppression seen in many cancer patients. Immune suppression by cancer has been well-documented in advanced cancer patients possessing a variety of malignancies. Correlation between immune suppression and poor prognosis has been extensively noted. Several means of tumor suppression of immune response are known. For example, a variety of tumor cells possess the ability to induce cleavage of the T cell receptor zeta (TCR-ζ) chain through a caspase-3 dependent manner. Since TCR-ζ is critical for signal transduction, host T cells become unable to respond to tumor antigens. Originally, the TCR-ζ cleavage was described in tumor bearing mice and subsequently in patients. The correlation between suppressed TCR-ζ and suppressed IFN-gamma production has been reported, implying functional consequences. The cause of TCR-ζ suppression has been attributed, at least in part, to reactive oxygen radicals produced by: A) The inflammatory activity occurring inside the tumor (it is well established that there is a constant area of necrosis intratumorally; B) Macrophages associated with the tumor; and C) Neutrophils activated directly by the tumor, or by the tumor associated macrophages.

Tumors are usually associated with macrophage infiltration, this is correlated with tumor stage and is believed to contribute to tumor progression by stimulation of angiogenesis. Cytokines such as M-CSF and VEGF produced by tumor infiltrating macrophages are essential for tumor progression to malignancy. In fact, tumors implanted into M-CSF deficient op/op mice (that lack macrophages) do not metastasize or become vascularized. Tumor-associated macrophages possess an activated phenotype and release various inflammatory mediators such as cyclo-oxygenase metabolites, TNF-□, and IL-6 which lead to increased levels of oxidative stress produced by host immune cells. In addition, tumor associated macrophages themselves produce large amounts of free radicals such as NO, OH, and H₂O₂. The high levels of macrophage activation in cancer patients is illustrated by high serum levels of neopterin, a tryptophan metabolite that is associated with poor prognosis.

In addition to oxidative stress elaborated by tumor associated macrophages, the presence of the tumor itself causes systemic changes associated with chronic inflammation. Erythrocyte sedimentation ration, C-reactive protein and IL-6 are markers of inflammatory stress used to designate progression of pathological immune diseases such as arthritis. Interestingly advanced cancer patients possess all of these inflammatory markers. Another marker of chronic inflammation is decreased albumin synthesis by the liver, this is also seen in cancer patients and is believed to contribute, at least in part, to cachexia. In addition, the inflammatory marker fibrinogen D-dimers is also higher in cancer patients as opposed to controls. Schmielau et al reported that in patients with a variety of cancers, activated neutrophils are circulating in large numbers. These neutrophils secrete reactive oxygen radicals such as hydrogen peroxide, which trigger suppression of TCR-ζ and IFN-gamma production. This was demonstrated by co-incubation of the neutrophils from cancer patients with lymphocytes from healthy volunteer. A profound suppression of TCR-ζ expression was seen. Evidence for the critical role of hydrogen peroxide was shown by the fact that addition of catalase suppressed TCR-ζ downregulation.

A simple method of assessing the number of circulating activated neutrophils was described in the same paper. This method involves collecting peripheral blood from patients, spinning the blood on a density gradient such as Ficoll, and collecting the lymphocyte fraction. While in healthy volunteers the lymphocyte fraction contained primarily lymphocytes, in cancer patients the lymphocyte fraction contained both lymphocytes and a large number of neutrophils. The reason why these neutrophils are present in the lymphocyte fraction is because activation alters their density so that they co-purify differently on the gradient.

A potential indication of the importance of activated neutrophils to cancer progression is provided by Tabuchi et al who show that removal of granulocytes from the peripheral blood of cancer patients resulted in reduced tumor size, unfortunately, the study was performed in only 2 patients. As a mechanism to compensate for immune over-activation, mediators of inflammation have immune suppressive properties. This is best illustrated in the immune suppression seen following immune hyperactivation such as in septic shock. Following the primary septicemia, patients are systemically immune compromised due to circulating immune suppressive factors that are released in response to the inflammatory stress. This suppression is termed compensatory anti-inflammatory response syndrome (CARS) and is associated with many opportunistic infections and deactivation. The clinical importance of CARS immune suppression is seen in that sepsis survivors show normal T-cell proliferation and IL-2 release, whereas those that succumb possess suppressed T cell responses. Interestingly immune suppressive mediators associated with CARS such as PGE2, TGF-□, and IL-10 are also associated with cancer-induced immune suppression.

The role of oxidative stress in sepsis-induced immune suppression was recently demonstrated in experiments where administration of antioxidants (ascorbic acid or n-acetylcysteine) to animals undergoing experimental sepsis blocked immune suppression. Another example of the potential for antioxidants to stimulate immune response in an inflammatory condition is in patients with Duke's C and D colorectal cancer who were administered of a daily dose of 750 mg of vitamin E for 2 weeks. This resulted in restoration of IFN-gamma and IL-2 production. The problem of uncontrolled inflammation is seen in sepsis. Although as a monotherapy n-acetylcysteine has little clinical effect, therapeutic administration of n-acetylcysteine results in suppression of the constitutively activated neutrophils seen in these patients. Administration of n-acetylcysteine to smokers results in suppression of markers of oxidative stress. Furthermore, oral n-acetylcysteine administration blocks angiogenesis and suppresses growth of Kaposi Sarcoma. Accordingly, a method of preparing the host for the TACE procedure includes administration of n-acetylcysteine at a concentration sufficient to decrease the tumor associated suppression of T cell activity. Such a concentration ranges between 1-10 grams per day, preferably 4-6 grams administered intravenously for a period of type sufficient to normalize production of IFN-gamma from PBMC of cancer patients upon ex vivo stimulation. One skilled in the art will understand that n-acetylcysteine is just one example of a compound suitable for reversion of oxidative-stress associated immune suppression. Numerous other compounds may be used, for example ascorbic acid, co-enzyme Q10 in combination with vitamin E and alpha-lipoic acid, genistein or resveratrol.

In some embodiments of the invention dendritic cells are utilized to induce an augmented immune response subsequent to TACE induced release of antigens. In other embodiments dendritic cells are administered close to the proximity of the TACE induced cell death. In one embodiment, DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a PlY kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mm L-glutamine, 10% of autologous, 50 ng/mL GM-CSF and 30 ng/mL IL-4. On day 6 immature dendritic cells are pulsed with tumor endothelial lysate or with ValloVax. Pulsing may be performed by incubation of lysates with dendritic cells or may be generated by fusion of immature dendritic cells with ValloVax cells.

Means of generating hybridomas or cellular fusion products are known in the art and include electrical pulse mediated fusion, or stimulation of cellular fusion by treatment with polyethylene glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of ValloVax pulsed dendritic cells is utilized as a polyvalent vaccine, whereas subsequent to administration antibody or t cell responses are assessed for induction of antigen specificity, peptides corresponding to immune response stimulated are used for further immunization to focus the immune response. Protocols useful for generation of dendritic cells have been previously used to generate immunity to a variety of tumors and are disclosed in the following which are incorporated by reference in melanoma, soft tissue sarcoma, thyroid, glioma, multiple myeloma, lymphoma, leukemia, as well as liver, lung, ovarian, and pancreatic cancer.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

1. A method of augmenting a vaccine induced immune response in a cancer patient, the method comprising:
a) immunizing a patient with a composition capable of inducing immunity against tumor endothelium; and
b) once damage to the tumor endothelium is accomplished, administering a vaccine preparation capable of stimulating immunity towards tumor specific antigens.

2. The method of claim 1, wherein the composition capable of inducing immunity against the tumor endothelium is a placental based endothelial cell vaccine.

3. The method of claim 1, wherein the composition capable of inducing immunity towards tumor endothelium is an endothelial progenitor cell.

4. The method of claim 3, wherein the endothelial progenitor cell is derived from a group of tissues comprising: a) placenta; b) umbilical vein endothelial cells; c) bone marrow cells; d) mobilized peripheral blood; and e) tumor derived endothelial cells.

5. The method of claim 4, wherein the endothelial progenitor cells are cultured under conditions capable of upregulating expression of markers found on tumor endothelial cells.

6. The method of claim 5, wherein the conditions capable of inducing expression of markers found on the tumor endothelial cells are selected from the group consisting of hypoxic conditions, acidotic conditions, and conditions associated with reduced tryptophan.

7. The method of claim 5, wherein the cells are treated in a manner to augment immunogenicity.

8. The method of claim 7, wherein augmentation of the cell immunogenicity is performed by treatment with an agent capable of augmenting expression of HLA antigens, wherein the agent capable of augmenting HLA expression is selected from the group consisting of interferon gamma, TNF-alpha, IL-33, IL-17, and valproic acid.

9. The method of claim 1, wherein the vaccine preparation capable of stimulating immunity towards tumor tissue is a whole cell, wherein the whole cell is comprised of at least one of allogeneic tumor cells and xenogeneic tumor cells.

10. The method of claim 1, wherein the vaccine preparation capable of stimulating immunity towards tumor tissue is a tumor specific antigen, wherein the tumor specific antigen is selected from a group comprising: ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

11. The method of claim 1, wherein the vaccine is an endogenous vaccine.

12. The method of claim 11, wherein the endogenous vaccine is tumor tissue induced to undergo cell death by means of at least one of localized radiation therapy, localized chemotherapy, localized hypothermia, localized immunotherapy, localized cryotherapy, and localized embolization.

13. The method of claim 1, wherein inducing an anticancer immune response in a cancer patient in need thereof is performed through the steps by:
admixing a concentration of immune stimulant with a clinically applicable localizing agent and a single or plurality of agents capable of causing localized cell death;
administering the combination directly into the tumor and/or arteries providing the tumor with blood supply; and
administering an embolizing agent in the proximity of the tumor and/or directly into the arteries providing the tumor with blood supply.

14. The method of claim 13, wherein the immune stimulant is a small molecule, a nucleic acid, a protein, or a combination thereof.

15. The method of claim 14, wherein the small molecule immune stimulant is selected from a group comprising of: muramyl dipeptide, thymosin, 7,8-disubstituted guanosine, imiquimod, detoxified lipopolysaccharide, isatoribine and alpha-galactosylceramide.

16. The method of claim 14, wherein the nucleic acid is selected from a group comprising of:
short interfering RNA targeting the mRNA of immune suppressive proteins, CpG oligonucleotides, Poly IC, unmethylated oligonucleotides, plasmid encoding immune stimulatory molecules, or chromatin-purified DNA.

17. The method of claim 13, wherein the protein is selected from one of the following compounds: IL-2, IL-7, IL-8, IL-12, IL-15, IL-18, IL-21, IL-23, IFN-gamma, TRANCE, TAG-7, CEL-1000, bacterial cell wall complexes, or LIGHT.

18. The method of claim 13, wherein the agent capable of causing cell death is a chemotherapeutic or radiotherapeutic agent, wherein the localizing agent is selected from the group consisting of an iodinated oil mixture and lipiodol, wherein the embolizing agent is selected from a group comprising of: Avitene, Gelfoam, Occlusin and Angiostat.

19. The method of claim 1, wherein the induction of tumor cell immunogenicity is performed through infection of tumor cells with an oncolytic virus.
